(19) United States
(12) Patent Application Publication
(10) Pub. No.: US 2016/0296133 A1
(43) Pub. Date: Oct. 13, 2016

(54) DIAGNOSTIC CATHETER SHAFT CONSTRUCTION AND MANUFACTURING METHOD

(71) Applicant: Oscor Inc., Palm Harbor, FL (US)

(72) Inventors: Thomas P. Osypka, Palm Harbor, FL (US); Allan Fuentes, Spring Hills, FL (US); Timothy A. Searfoss, New Port Richey, FL (US)

(21) Appl. No.: 15/093,294

(22) Filed: Apr. 7, 2016

Related U.S. Application Data
(60) Provisional application No. 62/144,411, filed on Apr. 8, 2015.

(51) Int. Cl. A61B 5/042; A61B 5/00; B29C 41/00; A61M 25/00

(52) U.S. Cl. CPC A61B 5/0422; A61M 25/005; A61M 25/0097; A61M 25/0012; A61B 5/6856; B29C 41/003; B29K 2077/00

(57) ABSTRACT

A shaft for a diagnostic catheter which includes a tubular core formed from a polyamide material, a braided layer surrounding the tubular core and formed from stainless steel strands, an inner layer surrounding the braided layer and formed from a polyimide material, and an outer layer surrounding the inner layer and formed from a polyether block amide material.

### CROSS-REFERENCE TO RELATED APPLICATIONS

The subject invention clams the benefit of priority from U.S. Provisional Patent Application Ser. No. 62/144,411 filed on Apr. 8, 2015, the disclosure of which is herein incorporated by reference in its entirety.

### BACKGROUND OF THE INVENTION

1. Field of the Invention

The subject invention is directed to cardiovascular catheters, and more particularly, to the construction of a shaft for a diagnostic catheter used in electrophysiological studies, such as mapping and the like, and to a method of manufacturing the same.

2. Description of Related Art

Cardiovascular electrode catheters are used to electrically stimulate and/or monitor the heart and, in some cases, to modify heart tissue through ablation. They are also often employed to perform diagnostic procedures such as mapping, and more generally for electrophysiology studies.

Such cardiovascular catheters typically comprise an elongated tubular catheter body with one or more electrodes mounted at the distal end of the catheter body. Wires extend from the electrodes through the catheter body to a connector at the proximal end of the catheter body. The connector can be plugged into an electrical stimulator and/or recorder or other source of electrical energy.

The catheter bodies of such cardiovascular catheters are typically made of an inner plastic tube surrounded by and reinforced with a braided stainless steel mesh. An outer plastic sleeve covers the reinforcing mesh. Such constructions generally provide high torsional stiffness, high resiliency and high flexibility. Indeed, the resulting catheter is usually more flexible in bending than is generally desirable.

One problem that has arisen with catheters constructed in this manner is that as it becomes more desirable to increase the diameter of the catheter lumen to accommodate a wider variety of instrumentation, it also becomes desirable to decrease the thickness of the walls of the tubes that form the catheter so that the device can still be easily delivered through the vasculature of a patient. It has been found however, that in thinner-walled catheters it is often more difficult to prevent the catheter from kinking as it is being pushed and pulled on its way through the vascular passage to the desired site in the body.

The subject invention provides a novel solution to this problem, allowing for the construction of a catheter having a relatively thin wall thickness while at the same time having a relatively small overall outer diameter that is below 4F in size.

### SUMMARY OF THE INVENTION

The subject invention is directed to a new and useful shaft construction for a diagnostic catheter which includes a tubular core formed from a polyamide material, a braided layer surrounding the tubular core and formed from stainless steel strands, an inner layer surrounding the braided layer and formed from a polyimide material, and an outer layer surrounding the inner layer and formed from a polyether block amide material.

Due to this construction, the shaft is able to have an outer diameter that is less than about 4F in size, and more preferably, the shaft has an outer diameter that is about 3.3F in size, while retaining the mechanical characteristic that are needed for pushing and pulling it on its way to a desired surgical site in the vasculature of a patient.

The tubular core of the shaft defines an interior lumen, and a handle assembly is operatively associated with a proximal end of the shaft, while an electrode assembly is operatively associated with a distal end portion of the shaft.

The subject invention is also directed to a diagnostic catheter that includes: a shaft having a tubular core formed from a polyimide material, a braided layer surrounding the tubular core and formed from stainless steel strands, an inner layer surrounding the braided layer and formed from a polyimide material, and an outer layer surrounding the inner layer and formed from a polyether block amide material; a handle assembly operatively associated with a proximal end portion of the shaft; and an electrode assembly joined to a distal end portion of the shaft.

Preferably, the electrode assembly includes a tubular shaft portion that is joined to the shaft of the catheter and a distal loop portion having a plurality of spaced apart electrodes. The tubular shaft portion of the electrode assembly is formed from a thermoplastic elastomer.

The subject invention is also directed to a method of manufacturing a diagnostic catheter shaft that includes the steps of extruding a polyimide tubular core, braiding a stainless steel layer over the polyimide tubular core, extruding a layer of polyimide material over the stainless steel braided layer, and molding a polyether block amide layer over the polyimide layer.

These and other features of the diagnostic catheter shaft of the subject invention and the manner in which it is manufactured and employed will become more readily apparent to those having ordinary skill in the art from the following enabling description of the preferred embodiments of the subject invention taken in conjunction with the several drawings described below.

### BRIEF DESCRIPTION OF THE DRAWINGS

So that those skilled in the art to which the subject invention appertains will readily understand how to make and use the diagnostic catheter shaft of the subject invention without undue experimentation, preferred embodiments thereof will be described in detail herein below with reference to certain figures, wherein:

FIG. 1 is a side elevational view of a diagnostic catheter constructed in accordance with a preferred embodiment of the subject invention;

FIG. 2 is a localized side elevational view of the distal portion of the diagnostic catheter shown in FIG. 1, illustrating the electrode assembly of the catheter;

FIG. 3 is a cross-sectional view of the catheter shaft taken along line 3-3 of FIG. 2, illustrating the layered construction of the catheter shaft;

FIG. 4 is a front end view of the diagnostic catheter portion of FIG. 1 illustrating the configuration of the electrode assembly; and

FIG. 5 is a localized side elevational view of the proximal portion of the diagnostic catheter shown in FIG. 1, illustrating the handle assembly of the catheter.

### DETAILED DESCRIPTION OF PREFERRED EMBODIMENTS

Referring now to the drawings, wherein like reference numerals identify similar structural features or aspects of the subject invention, there is illustrated in FIG. 1 a diagnostic catheter 10 constructed in accordance with a preferred embodiment of the subject invention for use, for example, in performing intra-cardiac electrophysiological mapping procedures for treating cardiac rhythm disorders. The catheter 10 may also be used for cardiac stimulation during electrophysiological studies.

Referring to FIG. 1, diagnostic catheter 10 includes an elongated shaft 12 having opposed proximal and distal end portions. A handle assembly 14, which is best seen in FIG. 5, is operatively associated with the proximal end portion of the shaft 12. The handle portion is adapted and configured for manipulating, controlling or otherwise operating the catheter 10 from a position that is remote from the surgical site. The total length of the catheter 10 is about 156 cm, with a usable length of about 146 cm.

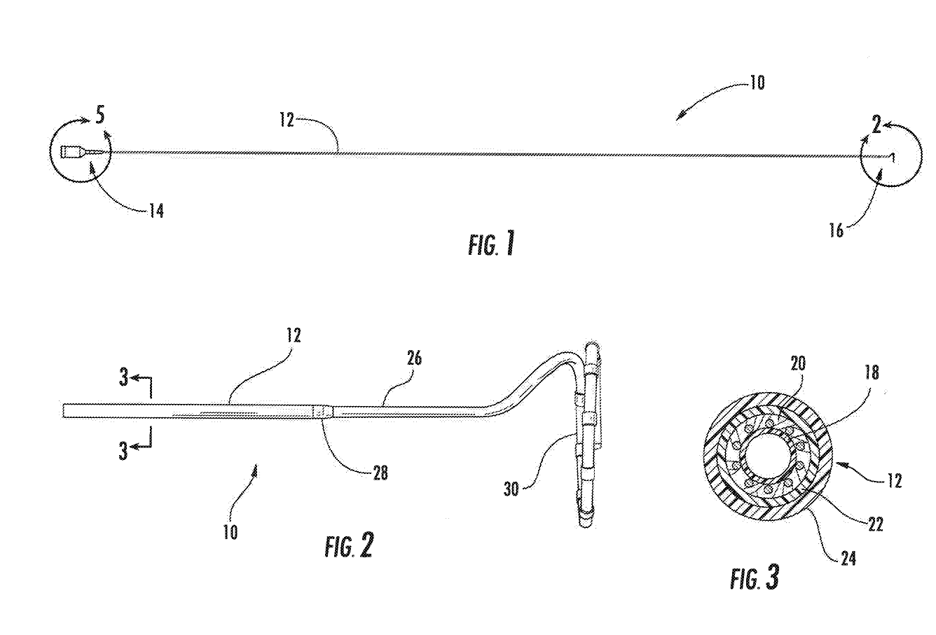

An electrode assembly 16 is operatively associated with the distal end portion of the elongated shaft 12 for performing diagnostic procedures such as mapping, and more generally for conducting electrophysiology studies directed at cardiac tissue or the like, and will be discussed in detail below with respect to FIGS. 2 and 4.

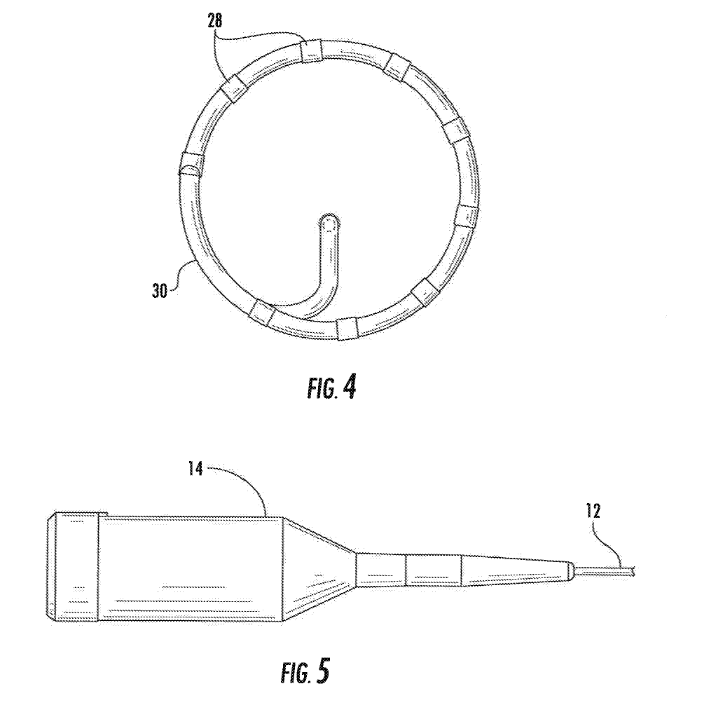

Referring to FIGS. 2 and 3, the elongated shaft 12 has a tubular core 18 formed from a polyamide material, and a braided layer 20 surrounding the tubular core 18. The braided layer 20 is preferably formed from stainless steel (e.g., 304SST) strands that are woven together in a conventional manner. A radially inner layer 22 formed from a polyimide material surrounds the braided layer 20. A radially outer layer 24 formed from a polyether block amide material surrounds the radially inner layer 22.

During the manufacturing process, the polyimide tubular core 18 is formed by extrusion. Then, the stainless steel braided layer 20 is disposed over the polyimide tubular core 18. Thereafter, the relatively thin surrounding layer 22 of polyimide material is extruded over the polyimide/braided assembly 18, 20. The outer polyether block amide layer 24 is then molded over the surrounding polyimide layer 22.

Referring now to FIGS. 2 and 4, the electrode assembly 16 includes a tubular shaft portion 26 that is joined to the shaft 12 of the diagnostic catheter 10 at a bonded joint 28 using conventional joining techniques known in the art. The electrode assembly 16 further includes a distal loop portion 30 having a plurality of spaced apart electrodes 32 for conducting an electrophysiological study such as mapping or the like.

The electrode assembly 16 can have a loop size of about between 15 mm and 20 mm. The number of electrodes can vary from about 8 or less to about 10 or more. The electrodes 32 are preferably spaced about 6 mm apart from one another and have an operative length of about 1 mm or more.

The tubular shaft portion 26 of the electrode assembly 16 is formed from a thermoplastic elastomer such as, for example, PA12 or the like, so that it is compatible with the materials from which the shaft 12 is constructed. Preferably, the tubular core 18 defines an interior lumen 34 to accommodate a guide wire or the like.

Those skilled in the art will readily appreciate that a polyether block amide or PEBA, such as that which is used to form the radially outer layer 24 is a high performance thermoplastic elastomer (TPE). It is also known under the tradename of PEBAX® (Arkema). It is a block copolymer obtained by polycondensation of a carboxylic acid polyamide (PA6, PA11, PA 12) with an alcohol termination polyether (Polytetramethylene glycol PTMG, PEG). One particular version of PEBAX® is 5533 SA 01 MED, which is designed to meet the stringent requirements of medical applications such as minimally invasive devices. It provides an excellent combination of properties such as kink resistance, low friction coefficient and superior

These materials are often used to replace common elastomers—thermoplastic polyurethanes, polyester elastomers, and silicones—for these characteristics: lower density among TPE, superior mechanical and dynamic properties (flexibility, impact resistance, energy return, fatigue resistance) and keeping these properties at low temperature (lower than −40° C.), and good resistance against a wide range of chemicals.

Those skilled in the art will also readily appreciate that the polyimide material from which the tubular core 18 and the radially inner layer 22 are formed is a polymer of imide monomers. It is a material that is lightweight, flexible and resistant to heat and chemicals, and is often used in the electronics industry for flexible cables, as an insulating film on magnet wire and for medical tubing. Examples of polyimide films include Apical, Kapton, UPILEX, VTEC PI, Norton TH and Kaptrex.

In accordance with a preferred embodiment of the subject invention, as a result of the materials selected for the layered construction of the shaft 12, catheter 10 has an outer diameter that is less than about 4F in size, and more preferably, the shaft 12 of catheter 10 has an outer diameter that is about 3.3F in size, 1.1 mm (0.043″), while retaining the mechanical characteristic that are needed for pushing and pulling the catheter on its way to a desired surgical site in the vasculature of a patient.

While the diagnostic catheter shaft of the subject invention has been shown and described with reference to preferred embodiments, those skilled in the art will readily appreciate that various changes and/or modifications may be made thereto without departing from the spirit and scope of the subject invention as defined by the appended claims.

What is claimed is:

1. A shaft for a diagnostic catheter comprising:
a) a tubular core formed from a polyimide material;
b) a braided layer surrounding the tubular core and formed from stainless steel strands;
c) an inner layer surrounding the braided layer and formed from a polyimide material; and
d) an outer layer surrounding the inner layer and formed from a polyether block amide material.

2. A shaft as recited in claim 1, wherein the shaft has an outer diameter that is less than about 4F in size.

3. A shaft as recited in claim 1, wherein the shaft has an outer diameter that is about 3.3F in size.

4. A shaft as recited in claim 1, wherein the tubular core defines an interior lumen.

5. A shaft as recited in claim 1, wherein a handle assembly is operatively associated with a proximal end of the shaft.

6. A shaft as recited in claim 1, wherein an electrode assembly is operatively associated with a distal end portion of the shaft.

7. A shaft for a diagnostic catheter comprising:
a) a tubular core formed from a polyimide material;
b) a braided layer surrounding the tubular core and formed from metal strands;
c) an inner layer surrounding the braided layer and formed from a polyimide material; and
d) an outer layer surrounding the inner layer and formed from a thermoplastic elastomer.

8. A shaft as recited in claim 7, wherein the braided layer is formed from stainless steel strands

9. A shaft as recited in claim 7, wherein the outer layer is formed from a polyether block amide material.

10. A shaft as recited in claim 7, wherein the shaft has an outer diameter that is less than about 4F in size.

11. A shaft as recited in claim 7, wherein the shaft has an outer diameter that is about 3.3F in size.

12. A shaft as recited in claim 7, wherein the tubular core defines an interior lumen.

13. A shaft as recited in claim 7, wherein a handle assembly is operatively associated with a proximal end portion of the shaft.

14. A shaft as recited in claim 7, wherein an electrode assembly is operatively associated with a distal end portion of the shaft.

15. A diagnostic catheter comprising:
a) shaft having a tubular core formed from a polyimide material, a braided layer surrounding the tubular core and formed from stainless steel strands, a radially inner layer surrounding the braided layer and formed from a polyimide material, and a radially outer layer surrounding the radially inner layer and formed from a polyether block amide material.
b) a handle assembly operatively associated with a proximal end portion of the shaft for controlling the catheter; and
c) an electrode assembly joined to a distal end portion of the shaft for conducting a mapping procedure.

16. A diagnostic catheter as recited in claim 15, wherein the shaft has an outer diameter that is less than about 4F in size.

17. A diagnostic catheter as recited in claim 15, wherein the tubular core defines an interior lumen.

18. A diagnostic catheter as recited in claim 15, wherein the electrode assembly includes a tubular shaft portion joined to the shaft of the catheter and a distal loop portion having a plurality of spaced apart electrodes.

19. A diagnostic catheter as recited in claim 18, wherein the tubular shaft portion of the electrode assembly is formed from a thermoplastic elastomer.

20. A method of manufacturing a diagnostic catheter shaft comprising:
a) extruding a polyimide tubular core;
b) braiding a stainless steel layer over the polyimide tubular core;
c) extruding a layer of polyimide material over the stainless steel braided layer; and
d) molding a polyether block amide layer over the polyimide layer.
